(19) United States
(12) Patent Application Publication
(10) Pub. No.: US 2019/0380870 A1
(43) Pub. Date: Dec. 19, 2019

(54) OCULAR GRAFT AND APPLICATOR

(71) Applicant: ChromoLogic LLC, Monrovia, CA (US)

(72) Inventors: Jawchyng Lormen Lue, Monrovia, CA (US); Juan Pablo Cardenas, Pasadena, CA (US); Ronald Robnett, Solana Beach, CA (US); Naresh Menon, Monrovia, CA (US); Evan Tsang, Pasadena, CA (US); Ed Burns, Temple City, CA (US)

(21) Appl. No.: 16/442,260

(22) Filed: Jun. 14, 2019

Related U.S. Application Data
(60) Provisional application No. 62/685,199, filed on Jun. 14, 2018.

(51) Int. Cl. A61F 9/00; A61K 9/00; A61F 9/007

(52) U.S. Cl. CPC A61F 9/0017; A61K 9/06; A61F 9/00781; A61K 9/0051

(57) ABSTRACT

An applicator includes a body defining a cavity configured to receive a container, a nozzle at an upper portion of the body, the nozzle comprising, an outer nozzle portion having a circular shape and defining a plurality of overflow channels, an inner nozzle portion having a circular shape and spaced from the outer nozzle portion by a circular channel, the inner nozzle portion including a ridge around a periphery of the inner nozzle portion and a cleft defining the ridge, the cleft and the ridge each having circular shapes, and a plurality of connectors extending through the channel and connecting the inner nozzle portion to the outer nozzle portion, wherein the overflow channels extend radially outward from the channel, and a plunger including a shaft and a base, the shaft being movable within the cavity and configured to guide the container toward the nozzle.

### CROSS-REFERENCE TO RELATED APPLICATION

The present application claims priority to and the benefit of U.S. Provisional Patent Application No. 62/685,199, filed on Jun. 14, 2018, the entire content of which is incorporated herein by reference.

### STATEMENT REGARDING FEDERALLY SPONSORED RESEARCH OR DEVELOPMENT

This invention was made with U.S. Government support under Contract W81XWH-17-C-0010. The U.S. Government has certain rights to this invention.

### BACKGROUND

### 1. Field

The present disclosure relates generally to the field of ocular grafts, circular applicators, and adhesive applicators for ocular grafts.

### 2. Description of Related Art

Corneoscleral injuries, such as corneal ruptures, are difficult to treat and require rapid treatment to prevent (or reduce the likelihood of) loss of an injured eye or loss of eyesight in the injured eye. When a cornea and/or sclera is ruptured, the eye cannot maintain interocular pressure (IOP) and eye fluids may then leak from the eye, which can lead to loss of the eye or eyesight in the injured eye.

Historically, in order to preserve an eye that experiences a corneal and/or scleral rupture, immediate surgery has been required. However, due to location of the injury, limited resources, and many other limiting factors, rapid surgery may not be feasible.

Accordingly, there is a need for a treatment of a ruptured cornea and/or sclera that substantially maintains IOP of the eye after the cornea is ruptured that can be applied safely and easily, particularly when immediate access to a hospital or surgeon is limited.

Various additional applications require controlled extrusion of a viscous fluid in a round pattern. For example, glue, grease, adhesive, lubricants, etc. may be applied in a circular pattern onto secondary devices such as O-rings, washers, bearings, joints, etc. These secondary devices may be rigid, such as those made of metals, or pliable, such as those made from plastic, rubber, silicone, Teflon, etc. As such, there is a need for an applicator that can extrude a viscous fluid in a circular shape, such as a continuous circular shape.

### SUMMARY

One or more embodiments of the present disclosure provide an ocular graft. One or more embodiments of the present disclosure provide an adhesive applicator for applying adhesive to a corneal graft. One or more embodiments of the present disclosure provide a method of gluing a corneal graft to an eye using an adhesive applicator kit.

According to one or more embodiments of the present disclosure, an applicator includes a body defining a cavity configured to receive a container, a nozzle at an upper portion of the body, the nozzle comprising, an outer nozzle portion having a circular shape and defining a plurality of overflow channels, an inner nozzle portion having a circular shape and spaced from the outer nozzle portion by a circular channel, the inner nozzle portion including a ridge around a periphery of the inner nozzle portion and a cleft defining the ridge, the cleft and the ridge each having circular shapes, and a plurality of connectors extending through the channel and connecting the inner nozzle portion to the outer nozzle portion, wherein the overflow channels extend radially outward from the channel, and a plunger including a shaft and a base, the shaft being movable within the cavity and configured to guide the container toward the nozzle.

In one or more embodiments, the body may include a barrel portion, and an inner wall of the barrel portion may have female threads and at least a portion of an outer wall of the plunger may have male threads, the male threads of the plunger mating with the female threads of the barrel portion.

In one or more embodiments, the applicator may further include a nut below the nozzle, the nut being in a clearance fit configuration with the body, and configured to resist rotation by the plunger when the plunger is rotated.

In one or more embodiments, the nut may include a stopple including a seal breaker at a lower portion of the stopple, the seal breaker having a first thickness at an uppermost portion of the seal breaker and a second thickness at a lowermost portion of the seal breaker, the first thickness being greater than the second thickness, and the seal breaker may define a stopple opening at a radial center of the stopple, the stopple opening being in fluid communication with the channel of the nozzle.

In one or more embodiments, an inner wall of the stopple may include female threads, the female threads being configured to mate with male threads of a container nozzle of the container.

In one or more embodiments, the nozzle may further include an inlet port in fluid communication with the channel and between the channel and the plunger along a longitudinal direction of the applicator, and the inlet port may have a diameter that is greater than or equal to an inner diameter of the outer nozzle portion.

In one or more embodiments, the body may include a plurality of vents defined in a top surface of the body, the vents being in fluid communication with the cavity.

In one or more embodiments, the body may include a barrel portion and a bayonet portion, the bayonet portion comprising a plurality of bayonet pins protruding from the bayonet portion, and the applicator may further include a cap, the cap defining a plurality of slots configured to mate with the bayonet pins.

In one or more embodiments, the cap may include a pad at a radial center thereof, the pad extending toward the nozzle when the cap is mated with the bayonet portion.

One or more embodiments of the present disclosure include a contact lens including an outer wall have a substantially continuous curvature, and an inner wall, the inner wall including a first portion having a first curve radius, a secondary portion having a second curve radius, and a peripheral portion having a third curve radius, the second curve radius being different from the first curve radius and the third curve radius.

In one or more embodiments, the second curve radius may be greater than the first curve radius.

In one or more embodiments, the third curve radius may be greater than the second curve radius.

In one or more embodiments, the contact lens may have a center thickness at an apex of the contact lens of between about 0.10 mm and about 0.14 mm.

One or more embodiments of the present disclosure are directed to a corneal graft and adhesive applicator kit, the kit including a contact lens including an outer wall have a substantially continuous curvature, and an inner wall, the inner wall including a first portion having a first curve radius, a secondary portion having a second curve radius, and a peripheral portion having a third curve radius, the second curve radius being different from the first curve radius and the third curve radius, an adhesive container including a body and an orifice, the adhesive container containing an adhesive, an adhesive applicator including a body defining a cavity configured to receive an adhesive container, a nozzle at an upper portion of the body, the nozzle including an outer nozzle portion having a circular shape and defining a plurality of overflow channels, an inner nozzle portion having a circular shape and spaced from the outer nozzle portion by a circular channel, the inner nozzle portion including a ridge around a periphery of the inner nozzle portion and a cleft defining the ridge, the cleft and the ridge each having circular shapes, and a plurality of connectors extending through the channel and connecting the inner nozzle portion to the outer nozzle portion, wherein the overflow channels extend radially outward from the channel, and a plunger including a shaft and a base, the shaft being movable within the cavity and configured to guide the adhesive container toward the nozzle, and an introducer including jaws and a handle.

In one or more embodiments, the handle of the introducer includes a first handle arm, a second handle arm, and a handle spring connecting the first handle arm and the second handle arm.

### BRIEF DESCRIPTION OF THE DRAWINGS

These and other features and aspects of embodiments of the present disclosure will be better understood by reference to the following detailed description, when considered in conjunction with the accompanying figures. The same reference numerals are used throughout the figures to reference like features and components.

FIG. 1 is a perspective view of an applicator according to one or more embodiments of the present disclosure.

FIG. 2 is a top view of the applicator of FIG. 1 according to one or more embodiments of the present disclosure.

FIG. 3 is a front elevational view of the applicator of FIG. 1 according to one or more embodiments of the present disclosure.

FIG. 4 is a side view of a portion of a body of the applicator of FIG. 3 according to one or more embodiments of the present disclosure.

FIG. 5 is a top view of a nozzle according to one or more embodiments of the present disclosure.

FIG. 6 is a cross-sectional view of the nozzle of FIG. 5 taken along the line 6-6 of FIG. 5.

FIG. 7 is a bottom view of a nut according to one or more embodiments of the present disclosure.

FIG. 8 is a schematic side view of the nut of FIG. 7 according to one or more embodiments of the present disclosure.

FIG. 9 is a schematic side view of a stopple according to one or more embodiments of the present disclosure.

FIG. 10 is a side elevational view of a plunger according to one or more embodiments of the present disclosure.

FIG. 11 is a top view of the plunger of FIG. 10 according to one or more embodiments of the present disclosure.

FIG. 12 is a perspective view of the applicator of FIG. 1 and a cap according to one or more embodiments of the present disclosure.

FIG. 13 is a bottom view of the cap of FIG. 12 according to one or more embodiments of the present disclosure.

FIG. 14 is a bottom perspective view of the cap of FIG. 13 according to one or more embodiments of the present disclosure.

FIG. 15 is a perspective view of an introducer according to one or more embodiments of the present disclosure.

FIG. 16 is a side view of an introducer according to one or more embodiments of the present disclosure.

FIG. 17 is a cross-sectional view of a lens according to one or more embodiments of the present disclosure.

FIG. 18 is a perspective view of an adhesive container according to one or more embodiments of the present disclosure.

FIG. 19A is a cross-sectional view of a method of applying an adhesive to a lens according to one or more embodiments of the present disclosure.

FIG. 19B is a cross-sectional view of a method of applying an adhesive to a lens according to one or more embodiments of the present disclosure.

FIG. 19C is a cross-sectional detailed view of a method of applying an adhesive to a lens according to one or more embodiments of the present disclosure.

FIGS. 20A-20B are schematic views of a method of adhering a lens to an eye according to one or more embodiments of the present disclosure.

### DETAILED DESCRIPTION

Embodiments of the present disclosure relate to a contact lens having multiple curvatures and being configured to be glued to an eye. According to some embodiments, a circular applicator is configured to extrude a fluid (e.g., a viscous fluid) in a ring-shaped extrusion. For example, a circular applicator may be utilized to dispense adhesive, glue, grease, lubricants, etc. The fluid is dispensed in a ring-shape (or a hollow circular shape) and may thus be easily applied to another surface where the other surface may also have a circular shape. In some example embodiments, an adhesive applicator is configured to extrude adhesive (e.g., glue) in a ring-shape. According to some embodiments, the adhesive is applied to the contact lens and the contact lens is adhered to an eye. The drawings depict some example embodiments for illustrative purposes only, and it will be apparent that modifications may be made without departing from the spirit and scope of the invention, and also that the present disclosure may also be used in other applications in the same or similar fields. Although relative terms such as “first,” “second,” “top,” “bottom,” “upper,” “lower,” and similar terms have been used herein to describe relative spatial relationships between elements, it is to be understood that these terms are intended to encompass different orientations of the various elements and components of the device in addition to the orientation depicted in the figures. Moreover, the figures contained in this application are not necessarily drawn to scale and various features may be exaggerated.

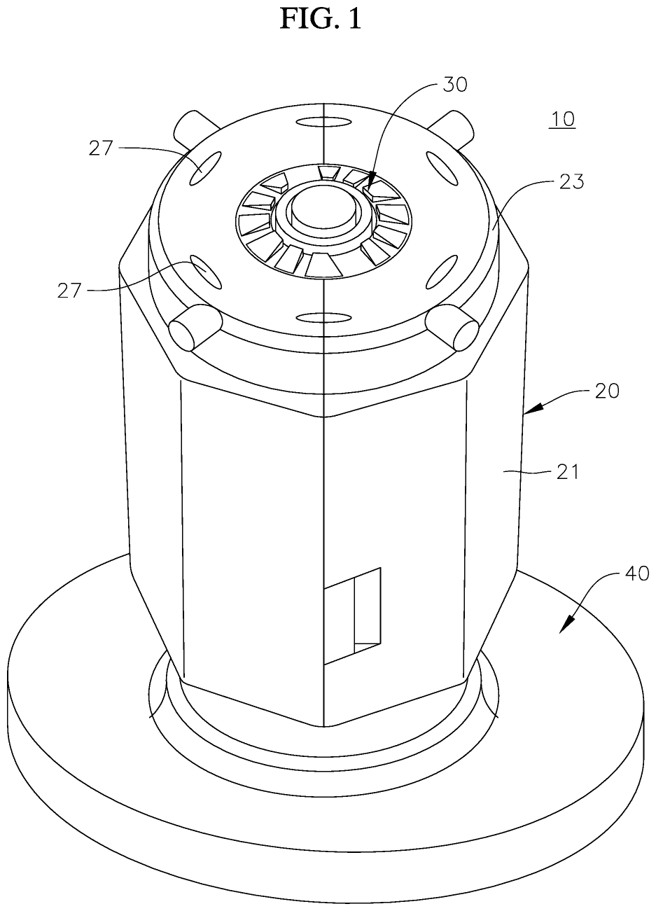

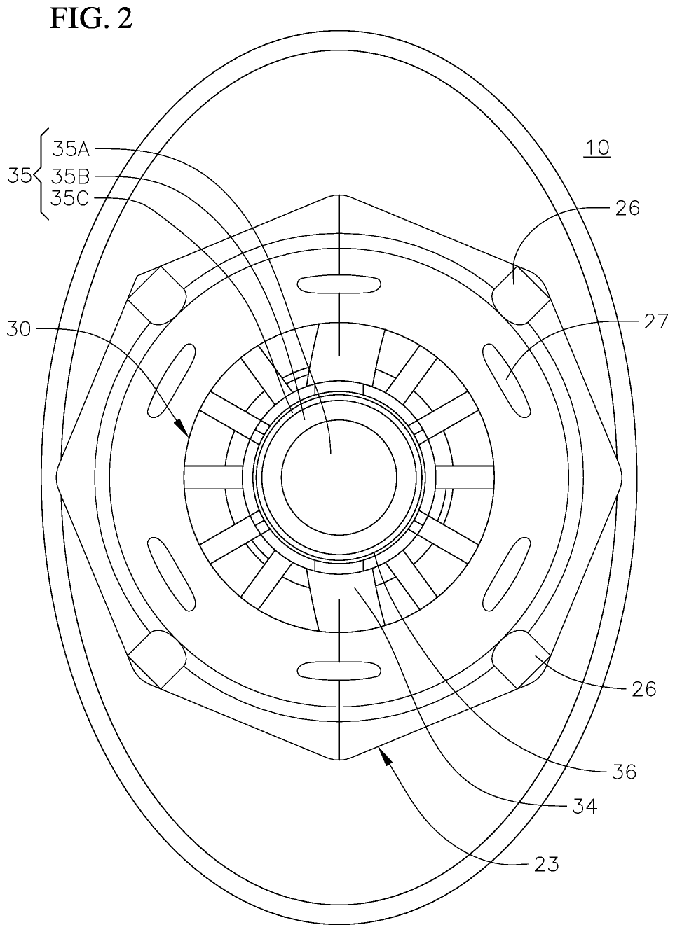

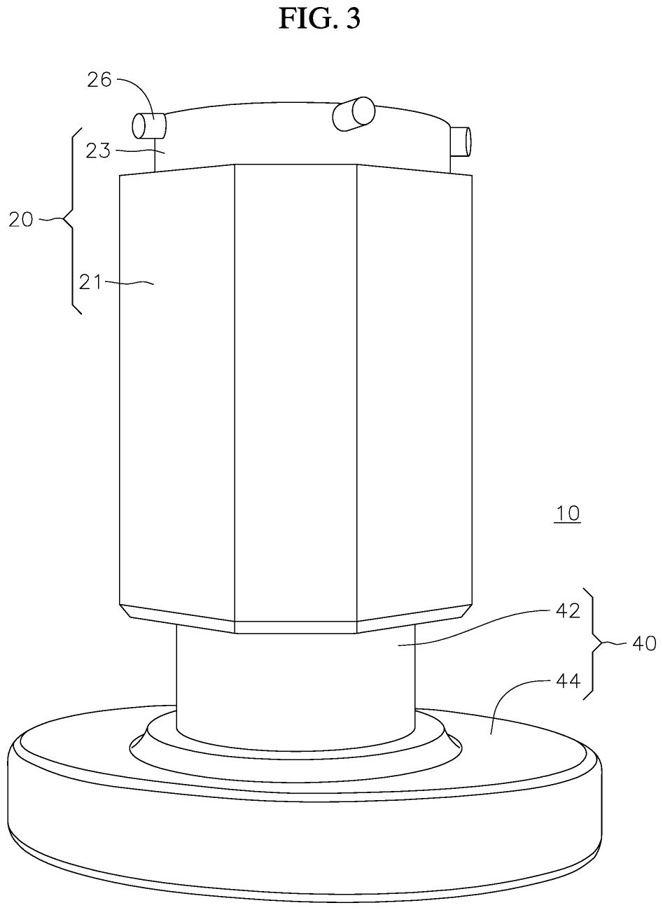

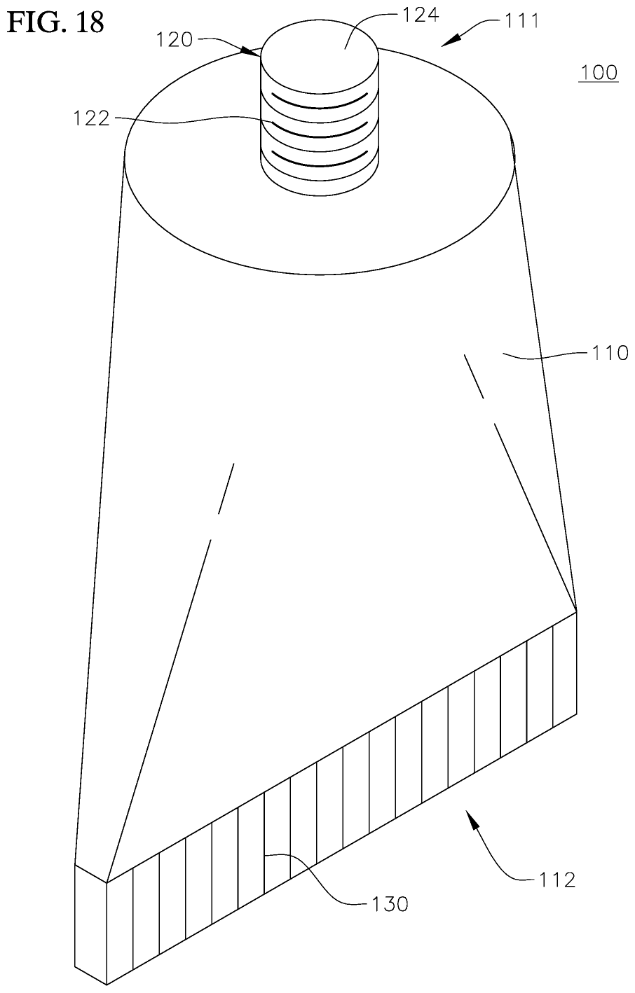

Referring now to an example embodiment illustrated in FIGS. 1-4, an applicator (e.g., an adhesive applicator) 10 includes a body 20, a nozzle 30, a nut 55, and a plunger 40. The body 20 defines a cavity 22 that is configured to receive an adhesive container 100 that contains an adhesive (e.g., glue). According to one or more embodiments, the adhesive container 100 may have a squeeze tube shape (or a collapsible tube shape), as is common for adhesives, artist's paint, toothpaste, ointments, etc. (see, e.g., FIG. 18). The adhesive container 100 may have a tubular body 110, an orifice 120 at a first end 111 of the body 110 and a crimped portion 130 at a second end 112 of the body 110 opposite to the first end 111. In some embodiments, the orifice 120 may include an adhesive container nozzle 122 that is threaded. Although reference is made to an adhesive container containing adhesive, it will be appreciated that other fluids may be contained in the container, such as lubricant, grease, glue, etc.

According to one or more embodiments, the body 20 may include a barrel portion 21 and a bayonet portion 23. As discussed further below, the barrel portion 21 may correspond to the cavity 22, and the bayonet portion 23 may house the nozzle 30. The barrel portion 21 may include an outer wall 21A and an inner wall 21B that defines the cavity 22. As discussed further below, in some embodiments, at least a portion of the inner wall 21B is threaded.

The barrel portion 21 may have any suitable shape. For example, the outer wall 21A may have a substantially octagonal shape. However, the present disclosure is not limited thereto, and the outer wall 21A may have a circular shape, an elliptical shape, a pentagonal shape, a hexagonal shape, etc. In some embodiments, the outer wall 21A may have at least one flat (or substantially flat) surface to enable a user to grip the body 20 more easily.

The body 20 may be integrally formed, or may be formed in multiple pieces that are configured to be assembled. For example, the body 20 may be formed as two halves, with a first half defining one or more recesses and a second half defining one or more clips configured to mate with the one or more recesses. In some embodiments, the recesses and the clips may be located at a lower portion of the barrel portion 21, for example at opposing vertices of the outer wall 21A of the barrel portion 21. The one or more clips may be configured to mate with the one or more recesses in an interference fit configuration such that the halves may be securely connected to each other and may be removable by applying outward pressure.

The body 20 further defines a plunger opening 28 at a lower portion of the barrel portion 21. The plunger opening 28 is configured to receive a shaft 42 of the plunger 40 and to allow for movement of the plunger 40 inside the cavity 22.

The body 20 further defines a nozzle opening 24 at an upper portion of the bayonet portion 23. The nozzle opening 24 surrounds the nozzle 30. The bayonet portion 23 may have a substantially circular shape and may include one or more bayonet pins 26 protruding from the bayonet portion 23. For example, the bayonet portion 23 may include four bayonet pins 26 at an outer wall 23A of the bayonet portion 23. The bayonet pins 26 may extend radially away from the bayonet portion 23. The bayonet portion 23 may define a plurality of vents 27 in an upper surface thereof. The vents 27 may define through-holes that provide fluid communication between the cavity 22 and outside of the body 20. The vents 27 may be formed outside of the nozzle opening 24.

An inner wall 23B of the bayonet portion 23 may define the nozzle opening 24. The nozzle opening 24 may be substantially circular. In some embodiments, the nozzle opening 24 may have a radially stepped shape, as discussed further below.

The inner wall 23B of the bayonet portion 23 may further define a nut opening 25 between the nozzle opening and the inner wall 21B of the barrel portion 21. The nut opening 25 may be configured to receive the nut 55 which receives the nozzle 122 of the adhesive container 100. The nut opening 25 may have one or more flat (or substantially flat) sides, which prevent the nut 55 from rotating in the nut opening 25. According to some embodiments, the nut opening 25 may have a hexagonal shape. The vents 27 may be formed outside of the nut opening 25 and may extend through the bayonet portion 23 along a longitudinal direction Z of the adhesive applicator 10 to provide fluid communication between the cavity 22 and the outside via the vents 27.

According to embodiments, the nozzle opening 24 and the plunger opening 28 are located at opposite ends of the adhesive applicator 10 along the longitudinal direction Z of the adhesive applicator 10.

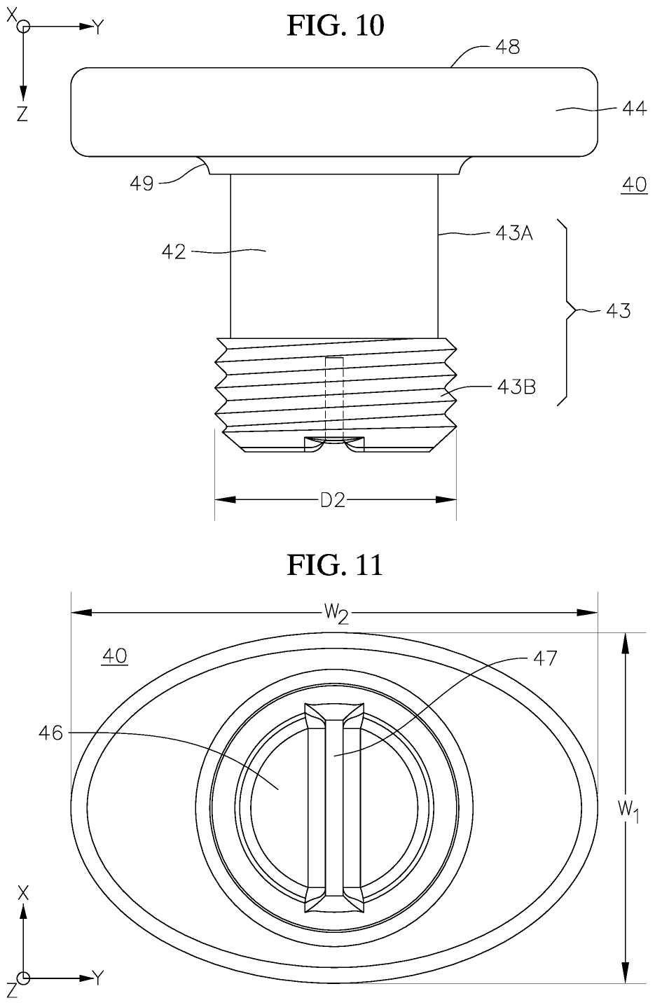

Referring to FIGS. 10-11, the plunger 40 may include the shaft 42 and a base 44. The shaft 42 is configured to be inserted through the plunger opening 28 into the cavity 22 of the body 20. The plunger opening 28 may have a first diameter D1 that is slightly greater than a second diameter D2 of the shaft 42, such that the shaft 42 may be inserted inside of the plunger opening 28 and is movable inside the body 20.

In some embodiments, an outer wall 43 of the shaft 42 may be substantially cylindrical. In some embodiments, at least a portion of the outer wall 43 of the shaft 42 may be threaded. For example, a lower portion 43A of the outer wall 43 of the shaft 42 adjacent (or near) the base 44 may be smooth (or substantially smooth) and an upper portion 43B of the outer wall 43 of the shaft 42 that is inserted into the cavity 22 may be threaded. For example, the upper portion 43B may have a male thread and at least a portion of the inner wall 21B of the barrel portion 21 may have a female thread. As such, the plunger 40 may be rotated, which drives at least a portion of the lower portion 43A of the shaft 42 in or out of the body 20 along the longitudinal direction Z. In some embodiments, the outer wall 43 of the shaft 42 and the inner wall 21B of the barrel portion 21 may both be substantially smooth, such that the plunger 40 may be actuated by applying pressure to the base 44 along the longitudinal direction Z to drive the at least a portion of the lower portion 43A of the shaft 42 in or out of the body 20. It will be appreciated that in embodiments where the outer wall 43 of the shaft 42 and the inner wall 21B of the barrel portion 21 include mating threads, the movement of the plunger 40 may be particularly precise by allowing for small, controlled advances based on rotation of the plunger 40. However, it will be appreciated that the plunger 40 may also be actuated linearly (e.g., similar to a piston).

The shaft 42 may include a top face 46 in a plane orthogonal to the longitudinal direction Z. In some embodiments, the top face 46 of the plunger 40 defines a groove 47. The groove 47 may have a length along a width direction X orthogonal to the longitudinal direction Z, and has a depth in the longitudinal direction Z. The groove 47 is configured to receive the crimped portion 130 of the adhesive container 100. The groove 47 may have any suitable depth in the longitudinal direction Z such that the groove 47 can receive and hold the adhesive container 100. According to one or more embodiments, the depth of the groove is substantially equal to a length of the crimped portion 130 of the adhesive container 100. The top face 46 (e.g., the groove 47) is configured to contact the adhesive container 100 when the adhesive container 100 is located in the cavity 22. In other words, when the adhesive container 100 is located (or positioned or stored) in the cavity 22, when the plunger 40 is driven toward the nozzle 30, the top face 46 (e.g., the groove 47) contacts the adhesive container 100 (e.g., the crimped portion 122 of the adhesive container 100). When the plunger is driven further toward the nozzle 30, the adhesive container 100 is driven toward the nozzle 30.

The base 44 may have any suitable shape such that a user may operate the plunger 40 and such that the adhesive applicator 10 may be stably supported by the base 44. For example, the base 44 may have a lower face 48 in a plane orthogonal to the longitudinal direction Z that is substantially flat. As such, the lower face 48 may be placed on a flat surface and the adhesive applicator 10 may be stably supported by the base 44. As such, it will be appreciated that the body 20 may be moved relative to the plunger 40 while the plunger 40 is stably positioned on the flat surface, or the body 20 may be held in a user's hand and the plunger 40 may be moved relative to the body 20. The base 44 may have a first width W1 along the width direction X and a second width W2 along a length direction Y, the length direction Y being orthogonal to the width direction X and the longitudinal direction Z. In some embodiments, the first width W1 and the second width W2 may each be greater than the second diameter D2 of the shaft 42 to allow for a more stable footing and ease of rotation of the body 20 about the plunger 40. Similarly, the base 44 may have any suitable thickness in the longitudinal direction Z such that the base 44 may provide a stable footing for the adhesive applicator 10.

In one or more embodiments, the plunger 40 further includes a stopper 49 between the shaft 42 and the base 44. The stopper 49 may form a step around the shaft 42. The stopper 49 may have a height in the longitudinal direction Z such that the plunger 40 is prevented from extending further into the body 20 when the body 20 contacts the stopper 49. In some embodiments, the stopper 49 may be 1/16th of the length of the shaft 42 to provide for complete insertion of the shaft 42 into the barrel portion 21 without over-extruding the adhesive from the adhesive container 100. The height of the stopper 49 may be determined according to an overall length of the adhesive container 100, which may be determined according to an amount of adhesive required to form a circular adhesive extrusion at a desired width on the lens 1. In some embodiments, the stopper 49 may be omitted.

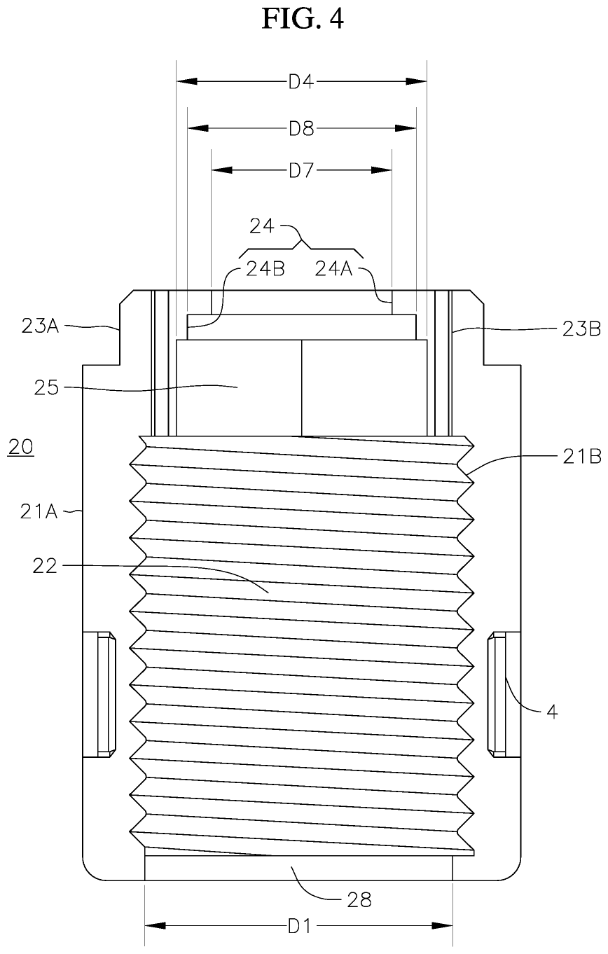

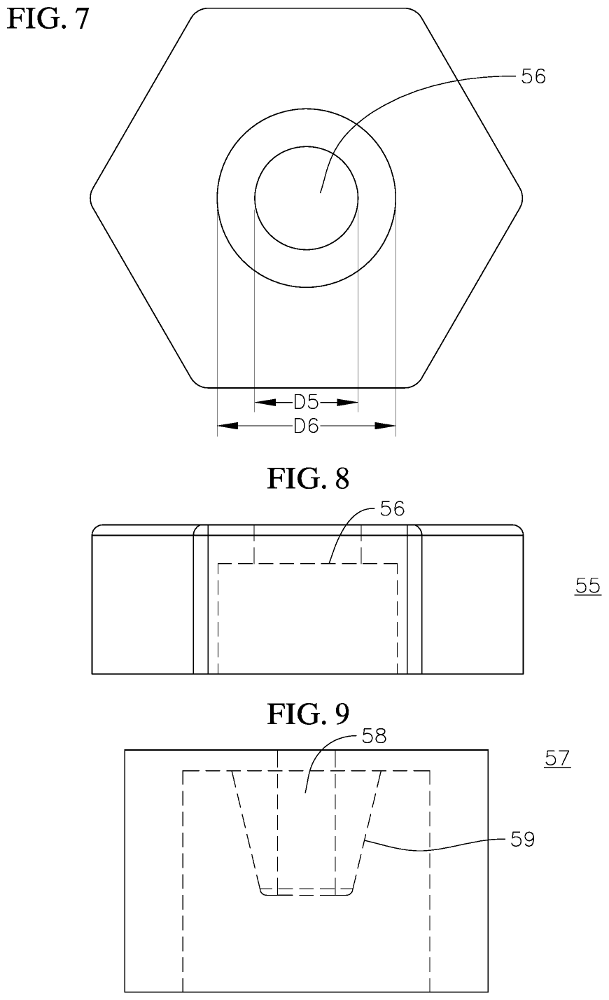

Referring to FIGS. 4, 7, 8, and 9, the nut 55 is inserted into the nut opening 25. The nut 55 may have a third diameter D3 that is substantially equal to or slightly less than a fourth diameter D4 of the nut opening 25 such that the nut 55 and the nut opening 25 are in a clearance fit configuration. The nut 55 may define a second nozzle opening 56 at a radial center thereof. The second nozzle opening 56 may have a stepped configuration such that the second nozzle opening 56 has a fifth diameter D5 at an upper portion of the nut 55 and a sixth diameter D6 at a lower portion of the nut 55. According to embodiments, the sixth diameter D6 is greater than the fifth diameter D5.

The second nozzle opening 56 is configured to receive a stopple 57. The stopple 57 includes a seal breaker 59 that defines a stopple opening 58 at a radial center of the stopple 57. The seal breaker 59 extends downwardly from a top surface of the stopple 57, and may have any suitable shape such that the seal breaker 59 punctures a film 124 on the nozzle 122 of the adhesive container 100. For example, the seal breaker 59 may have a conical cylindrical shape, such that a thickness of the seal breaker 59 at an uppermost portion of the seal breaker 59 is greater than a thickness of the seal breaker 59 at a lowermost portion of the seal breaker 59.

The stopple opening 58 allows the adhesive in the adhesive container 100 to flow through the stopple opening 58 when the adhesive applicator 10 is actuated. The stopple opening 58 may have any suitable diameter to allow the adhesive to flow through the stopple opening 58.

An inner surface of the stopple 57 includes female threads that correspond to male threads of the nozzle 122 of the adhesive container 100. As such, as the plunger 40 is rotated to drive the plunger 40 (e.g., the shaft 42) toward the nozzle 30, the film 124 is brought into contact with the seal breaker 59, which punctures the film 124, and the nozzle 122 of the adhesive container 100 is threaded into the stopple 57 in the nut 55. As discussed further below, the nut 55 then holds the adhesive container 100 in place, and as the plunger 40 is further driven toward the nozzle 30, adhesive is extruded through the punctured film 124 from the adhesive container 100 and through the stopple opening 58.

The nozzle 30 is designed to extrude adhesive as a continuous ring-shaped bead of adhesive. The continuous ring-shaped adhesive extrusion may be applied to a peripheral rim of a contact lens (e.g., an ocular graft) 1 to then be adhered to an eye, as discussed further below.

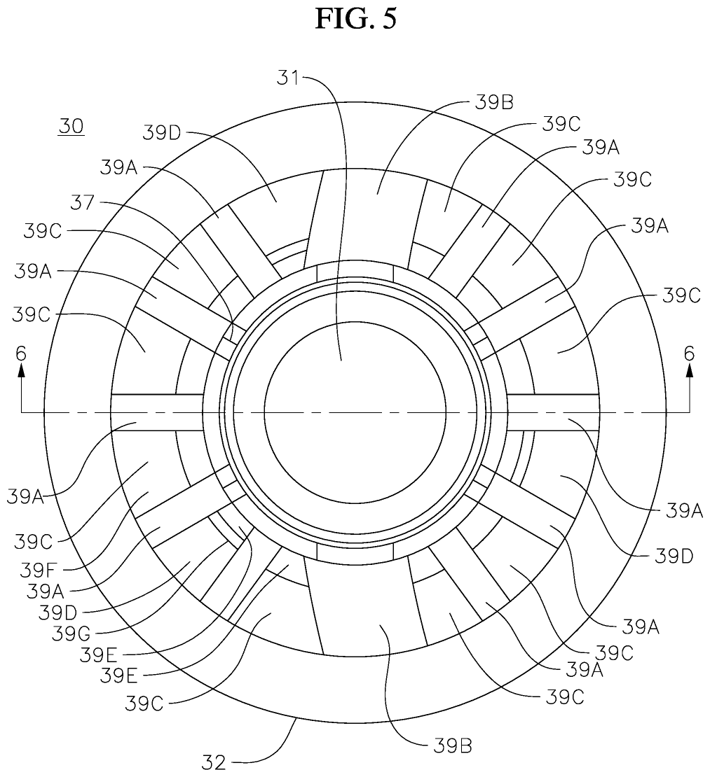

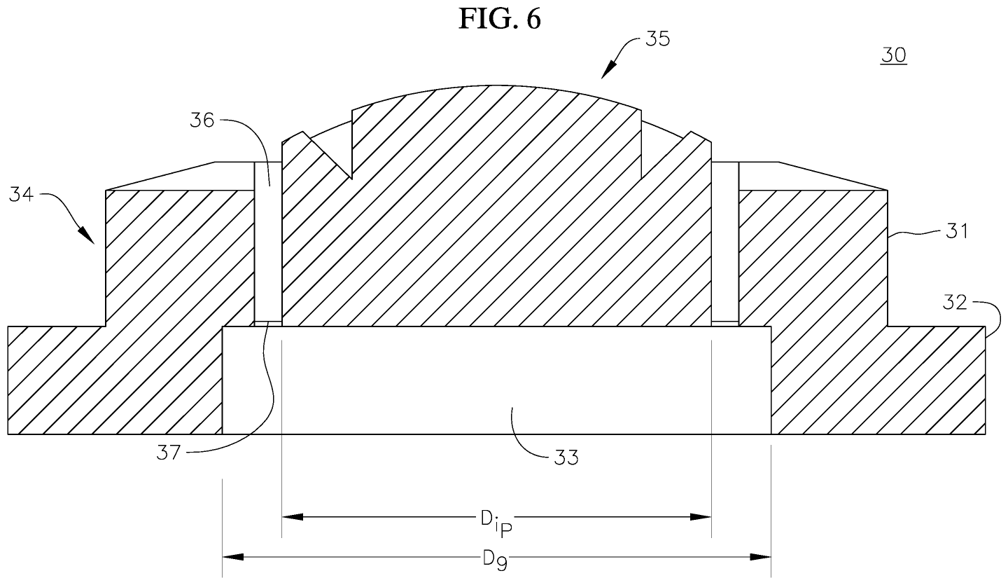

Referring to FIGS. 5 and 6, according to one or more embodiments of the present disclosure, the nozzle 30 includes a nozzle body 31 and a nozzle lip 32. The nozzle 30 has a circular shape with the nozzle lip 32 radially stepped from the nozzle body 31. The nozzle opening 24 defines a nozzle body opening 24A and a nozzle lip opening 24B. The nozzle body opening 24A has a seventh diameter D7 and the nozzle lip opening 24B has an eighth diameter D8. The eighth diameter D8 is greater than the seventh diameter D7 and is smaller than the fourth diameter D4 of the nut opening 25. The nozzle 30 fits into the nozzle opening 24 in a clearance fit configuration. For example, the nozzle lip 32 fits into the nozzle lip opening 24B in a clearance fit configuration and the nozzle body 31 fits into the nozzle body opening 24A in a clearance fit configuration according to embodiments. Because the seventh diameter D7 of the nozzle body opening 24A is smaller than the eighth diameter D8 of the nozzle lip opening 24B (and the nozzle body 31 therefore has a smaller outer diameter than an outer diameter of the nozzle lip 32), the nozzle lip opening 24B forms a stop that prevents the nozzle 30 from being removed through the nozzle opening 24.

According to embodiments, the nozzle lip 32 is located above the nut 55 and has a ring shape. For example, an inner diameter of the nozzle lip 32 may be less than the outer diameter of the nozzle body, but greater than the fifth diameter D5 of the second nozzle opening 56 defined in the nut 55.

According to one or more embodiments, the nozzle body 31 comprises an outer portion 34 and an inner portion 35. The outer portion 34 and the inner portion 35 are separated by a channel 36 surrounding the inner portion 35 and having a circular shape. A plurality of supports 37 may be formed in the channel 36 at a lower portion thereof to connect the outer portion 34 and the inner portion 35 to each other. The channel 36 has a ring shape and extends along the longitudinal direction Z to allow for passage of adhesive upward through the channel 36.

The inner portion 35 may have a cylindrical shape with a rounded upper surface 35A. The inner portion 35 has a ninth diameter D9. The ninth diameter D9 is determined according to a maximum inner diameter of the adhesive ring to be extruded through the nozzle 30. According to some embodiments, the ninth diameter D9 may be about 8 mm. As discussed further below, the rounded upper surface 35A may be rounded to generally correspond to a curvature of the lens 1. When the lens 1 is located over the nozzle 30, a gap 38 is formed between the inner portion 35 and the lens 1.

The inner portion 35 may further include a cleft 35B near a periphery of the inner portion 35. The cleft 35B is offset from an edge of the inner portion 35 such that the cleft 35B defines a ridge 35C at the edge of the inner portion 35, as illustrated in FIG. 6. The cleft 35B has a sufficient depth such that the cleft 35B prevents adhesive from flowing toward a center of the lens 1 during application of the adhesive due to a capillary effect of the adhesive. In some embodiments, the cleft 35B may have a cleft width Wcl of about 0.9 mm and an angle θ of 45 degrees between walls of the cleft 35B. The cleft width Wcl and the angle θ can be any suitable dimensions such that the cleft 35B prevents a capillary draft effect of the adhesive when the adhesive is applied to the lens 1.

The channel 36 and the ridge 35C may have suitable widths such that a predetermined amount of adhesive is applied and maintained on the lens 1. For example, the channel 36 may have a channel width Wc of about 0.325 mm to about 0.5 mm, and the ridge 35C may have a ridge width Wr of about 0.4 mm. As such, adhesive in the amount of between about 2.75 mg and about 12.72 mg may be applied to the lens 1 while forming a continuous adhesive ring. According to some embodiments, 8 mg of glue may be dispensed on the lens 1.

The nozzle lip 32 defines an inlet port 33 in fluid communication with the channel 36. For example, an inner wall of the nozzle lip 32 may define the inlet port 33. The inlet port 33 provides a manifold for adhesive to collect prior to extrusion and to provide an even extrusion of adhesive through the channel 36. The inlet port 33 has an inlet port diameter Dip that is greater than or equal to the sum of the ninth diameter D9 of the inner portion 35 and two times the channel width Wc. In other words, the inlet port 33 extends at least as far as the channel 36 along a radial direction of the nozzle 30 so that the adhesive collects below the channel 36 prior to being extruded through the channel 36.

Referring to FIG. 5, the outer portion 34 may define a plurality of overflow channels 39A, a plurality of introducer ways 39B, a plurality of nozzle lands 39C, and a plurality of locating tabs 39D. The overflow channels 39A extend from an inner diameter of the outer portion 34 to an outer diameter of the outer portion 34. The overflow channels 39A allow excess adhesive to flow outwardly away from a center of the lens 1. The overflow channels 39A allow excess adhesive to be pushed therethrough in a fixed space between the nozzle 30 and the lens 1. As a result, the volume/amount of adhesive in the defined space may be maintained (or consistent) regardless of the amount of the adhesive dispensed and even if excess adhesive is dispensed through the nozzle 30.

The overflow channels 39A may have a sufficient depth such that the overflow channels 39A are below the lens 1 when the lens 1 is on the nozzle 30 to prevent wicking of adhesive on an upper surface of the lens 1 during adhesive extrusion, which could otherwise occur due to adhesive and cohesive forces. For example, the overflow channels may have a depth of 0.5 mm. The overflow channels 39A may have a substantially constant width. In other words, a width of each of the overflow channels 39A at the inner diameter of the outer portion 34 may be equal (or substantially equal) to a width of the respective overflow channel 39A at the outer diameter of the outer portion 34. The utilization of multiple overflow channels 39A allows for robustness of adhesive application by providing for adhesive overflow and even dispensing of adhesive. For example, in some embodiments, ten overflow channels 39A may defined in the outer portion 34. In some embodiments, the overflow channels 39A may have a width of 1 mm.

The outer portion 34 may also include the nozzle lands 39C and the locating tabs 39D. The nozzle lands 39C also extend from the inner diameter of the outer portion 34 to the outer diameter of the outer portion 34. The nozzle lands 39C and the locating tabs 39D define the overflow channels 39A. In other words, the overflow channels 39A are defined between adjacent ones of the nozzle lands 39C and/or the locating tabs 39D. The nozzle lands 39C and the locating tabs 39D each have a raised portion 39e at the inner diameter of the outer portion 34 that extends radially from the inner diameter of the outer portion 34, and a sloping portion 39f that extends downwardly toward the outer diameter of the outer portion 34. The locating tabs 39D further include a raised portion 39g that extends upwardly from the raised portion 39e.

According to embodiments, the outer portion 34 includes three or more of the plurality of locating tabs 39D. The locating tabs 39D may be spaced apart from each other around the outer portion 34. For example, when the outer portion 34 includes three locating tabs 39D, the locating tabs 39D may be positioned at approximately 60 degrees, 180 degrees, and 240 degrees. The raised portions 39g of the locating tabs 39D may form a tripod. The raised portions 39g of the locating tabs 39D may support the lens 1 when the lens 1 is on the nozzle 30, may space the lens 1 from the overflow channels 39A and the raised portions 39f, and may reduce the likelihood that the lens 1 may slip or move radially during extrusion of the adhesive. The combination of the nozzle lands 39C and the locating tabs 39D may further reduce the likelihood of a capillary effect when the adhesive is extruded through the nozzle 30. According to embodiments, the lens 1 may contact at least a portion of the locating tabs 39D and at least a portion of the nozzle lands 39C when the lens 1 is located on the nozzle 30.

The outer portion 34 may further include the pair of introducer ways 39B extending from the inner diameter of the outer portion 34 to the outer diameter of the outer portion 34. In some embodiments, the introducer ways 39B face each other at the inner diameter of the outer portion 34 (e.g., are 180 degrees from each other), and the overflow channels 39A are formed between adjacent ones of the nozzle lands 39C and/or the locating tabs 39D around the outer portion 34. In some embodiments, the outer portion 34 may define ten overflow channels 39A, and five overflow channels 39A may be formed between the introducer ways 39B on each side of the introducer ways 39B. The introducer ways 39B are configured to accommodate jaws 210 of an introducer 200 which clasps the lens 1 after the adhesive is applied, discussed further below. A width of the introducer ways 39B and an internal radius of the introducer ways 39B may be determined such that the introducer ways 39B provide a clearance fit with the jaws 210 of the introducer 200 when the introducer is used to clasp the lens 1. The width of the introducer ways 39B may be greater than the width of the overflow channels 39A to reduce the likelihood of overflow of adhesive through the introducer ways 39B.

When the lens 1 is placed above the nozzle 30, the gap 38, the continuous adhesive ring, and the nozzle 30 provide a natural air pressure that resists the adhesive to flow inward during adhesive extrusion.

According to embodiments, the amount of adhesive dispensed onto the lens 1 is in a range of from about 2.75 mg to about 12.72 mg in order to provide a central clearing zone on the lens 1 when the lens 1 is applied to the cornea, while still providing a sufficient amount of adhesive to form a continuous ring when extruded onto the lens 1. The central clearing zone prevents the adhesive from obstructing central visual field and from entering the cornea lesion, or reduces the likelihood thereof. A continuous ring around the lens 1 allows the adhesive to create a tight seal on the cornea.

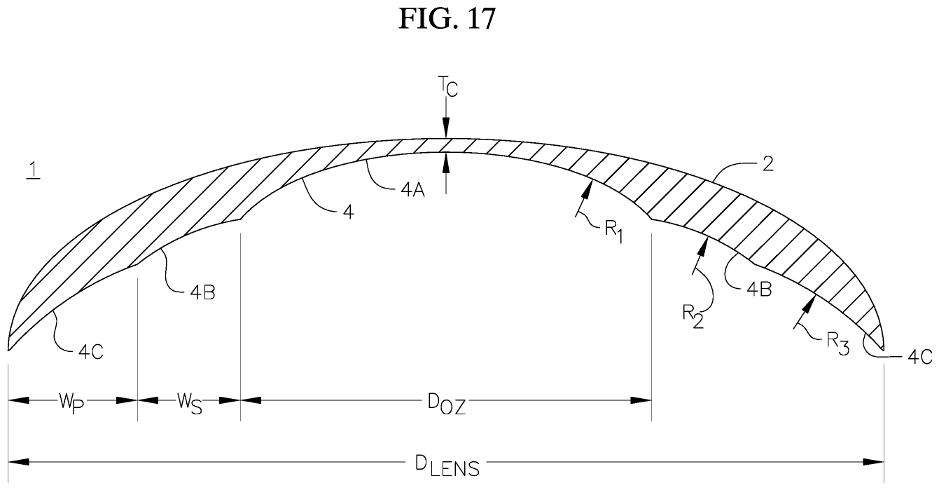

Referring to FIG. 17, according to one or more embodiments of the present disclosure, the lens (or the ocular graft) 1 may have a tri-curve design. The lens 1 includes an outer wall 2 and an inner wall 4. The outer wall 2 may have a substantially continuous curvature, similar to a conventional contact lens as is known in the art.

The inner wall 4 of the lens 1 may have three curvatures, as shown in FIG. 17. The inner wall 4 of the lens 1 may have a first portion (or a central portion) 4A at a central portion thereof, a second portion (or secondary portion) 4B at a periphery of the first portion 4A, and a third portion (or peripheral portion) 4C at a periphery of the second portion 4B. The first portion 4A may have a first curve radius R1 and a first portion diameter Doz which generally corresponds to an optic zone of an eye. For example, the first curve radius R1 may be between about 7.4 mm and about 8.2 mm to correspond to the natural curvature/shape of a human cornea. The first portion diameter Doz may be in a range of about 8.0 mm to about 8.4 mm to provide for clear, central vision through the first portion 4A of the lens 1.

The second portion 4B has a secondary curve radius R2 and a secondary portion width Ws. The secondary curve radius R2 is greater than the first curve radius R1. In some embodiments, the secondary curve radius R2 may be between about 9.4 mm and about 9.6 mm. The secondary portion width Ws may be between about 0.6 mm and about 0.8 mm. The secondary portion width Ws may be any suitable length to provide proper spacing between the first portion 4A and the third portion 4C.

The third portion 4C has a peripheral curve radius R3 and a peripheral portion width Wp. The peripheral curve radius R3 is greater than the secondary curve radius R2. In some embodiments, the peripheral curve radius R3 may be between about 10.4 mm and about 10.6 mm. The peripheral portion width Wp may be between about 0.4 mm and about 0.6 mm. The peripheral curve radius R3 and the peripheral portion width Wp may be any suitable length and radius to receive and accommodate the adhesive that is extruded onto the lens 1.

In more detail, the lens 1 is designed such that the adhesive is applied only to the peripheral portion 4C, which is spaced apart from the first portion 4A (that is located above the optic zone of an eye when the lens is adhered to the eye) via the secondary portion 4B. In one embodiment intended as a corneal graft, the lengths of the first portion diameter Doz, the secondary portion width Ws, and the peripheral portion width Wp are determined such that on the peripheral portion 4C, to which the adhesive is applied, the adhesive is located inside of the limbus region of an eye and the adhesive is applied only to the cornea. A lens diameter Dlens of the lens 1 is equal to a sum of the first portion diameter Doz, 2 of the secondary portion widths Ws, and 2 of the peripheral portion widths Ws. According to embodiments, the lens diameter Dlens is between about 10.50 mm and about 10.70 mm. As such, the lens diameter Dlens is smaller than an inner diameter of the limbus region of a human eye (or is smaller than an outer diameter of the cornea) to avoid damage to limbal cells in the limbus region of the eye when the lens 1 is glued to the eye. An edge portion of the lens 1 may be tapered to enhance comfort for the wearer. It will be appreciate that grafts of similar geometries may additionally be used for injuries to the sclera of the eye or of eyes of other creatures where the overall shape of the eye is different.

It will be appreciated that the first curve radius R1, the secondary curve radius R2, and the peripheral curve radius R3, do not have co-located radii, providing the lens 1 with radial symmetry and a smooth curvature at the outer wall 2. Further, as illustrated in FIG. 17, the lens 1 has a nonuniform thickness. The lens 1 may have a center thickness Tc at an apex of the lens (or at the radial center) of between about 0.10 mm and about 0.14 mm. The center thickness Tc provides high oxygen transmissivity through the lens 1 at the cornea while maintaining mechanical strength of the lens 1. The lens may have a power of −2.00±0.25 D, such that the center thickness Tc is approximately 0.12 mm.

According to embodiments, the lens 1 may be formed of a material that allows for oxygen permeability to allow for a proper seal when the lens 1 is adhered to the cornea, but to allow for oxygen to pass through the lens 1 to the cornea. The lens 1 may be substantially transparent (or have a low opacity), so that a user may be able to monitor adhesive application to the lens 1 and so that persons may be able to see through the lens 1 when it is adhered to the cornea or sclera, for example, for a doctor to monitor whether the eye has healed after adhering the lens 1. According to embodiments, the lens 1 is a hard contact lens. However, the present disclosure is not limited thereto, and any suitable contact lens may be used, such as soft contact lenses, scleral lenses, etc.

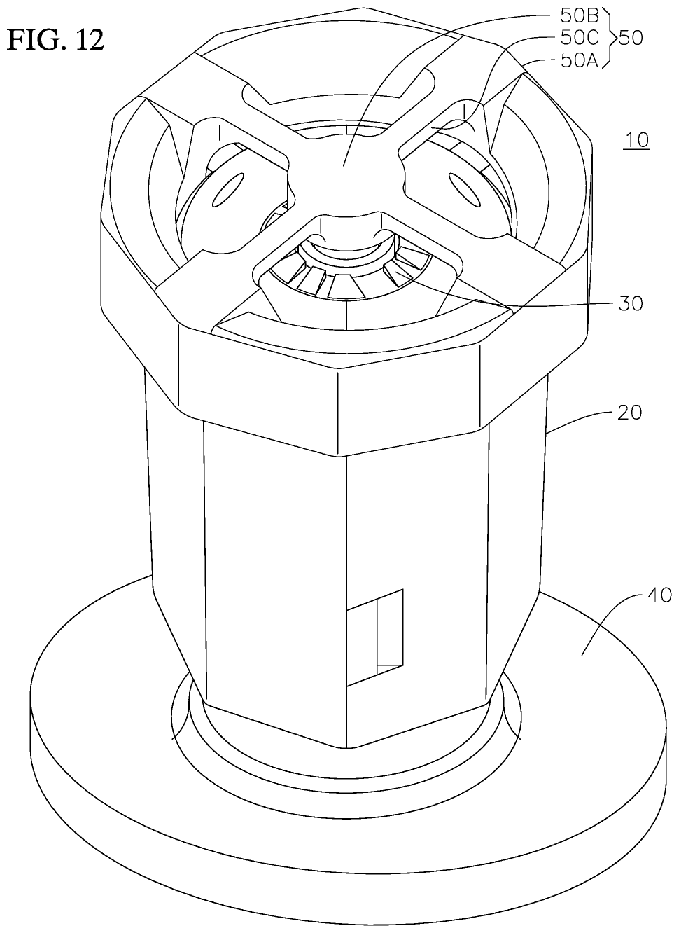

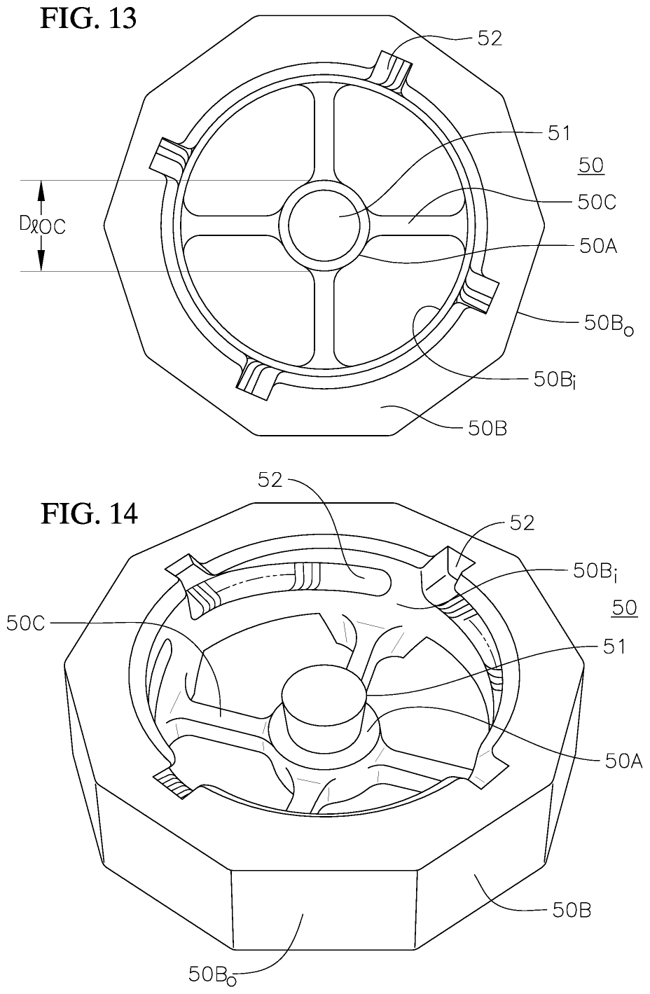

Referring to FIGS. 12-14, according to one or more embodiments of the present disclosure, the adhesive applicator 10 may further include a cap (or a lid) 50. According to embodiments, the cap 50 has a center portion 50A and an outer portion 50B, which are connected to each other by a plurality of connectors 50C.

The center portion 50A may have a substantially flat upper surface and a grooved lower surface that is configured to receive a pad 51 and to hold the pad 51 in an interference fit configuration. According to some embodiments, the pad 51 may be a foam material, such as polyurethane foam. However, the present disclosure is not limited thereto, and any suitable material may be used for the pad 51 such that it can be firmly held in the center portion 50A and will not scratch or otherwise damage the lens 1 when it contacts the lens 1. In some embodiments, the pad 51 and the groove in the lower surface of the center portion 50A may each have a diameter equal to 6 mm. However, the present disclosure is not limited thereto, and the groove and the pad 51 may have any suitable diameter such that the diameter of the pad 51 is less than an inner diameter of the ring of adhesive produced by the adhesive applicator 10. As such, a user may be able to view the adhesive as it is extruded onto the lens 1, even when the pad 51 is contacting the lens 1.

The center portion 50A may have a substantially round shape and a lid center outer diameter Dloc. The lid center outer diameter Dloc is greater than the diameter of the pad 51. In some embodiments, the lid center outer diameter may be 10 mm.

According to embodiments, the center portion 50A is connected to the outer portion 50B by the connectors 50C. In some embodiments, the cap 50 includes four connectors 50C extending radially from the center portion 50A and at right angles relative to each other. The connectors 50C may have any suitable width such that the center portion 50A is stably connected to the outer portion 50B and such that a user's vision is not substantially obstructed when looking through the cap 50. For example, when the lens 1 is located under the cap 50, the connectors 50C will at least partially obstruct the view of the lens 1 located underneath. However, a width of the connectors 50C may be reduced so that the lens 1 (and the adhesive ring applied thereto) are at least partially visible when the cap 50 is on the adhesive applicator 10. For example, in some embodiments, the connectors 50C may each have a width of 2 mm.

The cap 50 is configured to mate with the bayonet portion 23 of the body 20. For example, the outer portion 50B of the cap 50 includes one or more slots 52 configured to mate with the bayonet pins 26 of the bayonet portion 23. The outer portion 50B of the cap 50 may have any suitable shape to allow for mating with the bayonet portion 23 and to allow a user to grip the outer portion 50B and twist the outer portion 50B. For example, the outer portion 50B may have an inner wall 50Bi that is substantially circular, and an outer wall 50Bo that has at least one flat surface for gripping. For example, the outer wall 50Bo may have a decagonal shape.

The slots 52 are each defined at the inner wall 50Bi. Each of the slots 52 has a vertical portion that extends from a bottom face of the cap 50 and that allows for the cap 50 to be aligned with the bayonet pins 26 and pushed downwardly onto the bayonet pins 26 and the bayonet portion 23. The slots 52 also have sloping portions that extend along the inner wall 50Bi and have a depth in a radial direction of the outer portion 50B. In other words, the slots 52 are defined in the inner wall 50Bi and have a depth extending toward the outer wall 50Bo. The inner wall 50Bi may have any suitable diameter such that the slots 52 and the bayonet pins 26 mate in a clearance fit with each other. Similarly, the slots 52 may have any suitable depth such that the slots 52 and the bayonet pins 26 mate in a clearance fit with each other.

According to one or more embodiments, the sloping portions of the slots 52 may have any suitable length such that cap 50 is removed or secured with one quarter turn or less, allowing for removal of the cap 50 without the need to reposition the adhesive applicator 10, which reduces the likelihood of shifting the lens 1 when it is located under the cap 50 and/or smearing the adhesive after is applied on the lens 1.

An adhesive applicator kit may include the adhesive applicator 10, the lens 1, the adhesive container 100, and an introducer 200. The introducer 200 is similar to a “cross” style ophthalmic tweezer. The jaws 210 of the introducer 200 are in a tension position when the introducer 200 is not in use, and applying pressure to handle arms 222 of the introducer 200 moves the jaws 210 wider apart. The introducer 200 is configured to lightly grip the lens 1 after adhesive has been applied to the lens 1 using the adhesive applicator 10 and to allow for movement of the lens 1 from the adhesive applicator 10 to an eye. The introducer 200 provides light tension on the lens 1 without bending (or substantially bending) the lens 1.

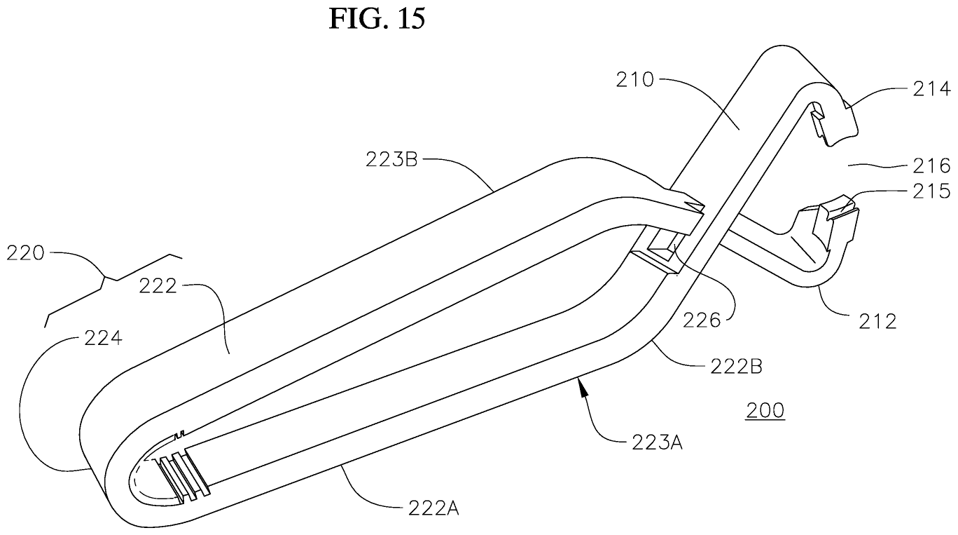

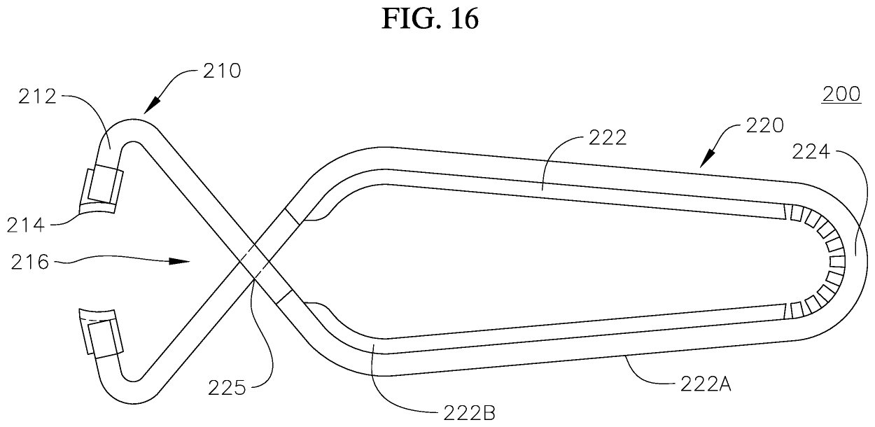

Referring to FIGS. 15-16, according to one or more embodiments, the introducer 200 includes the jaws 210 and a handle 220 including the handle arms 222 and a handle spring 224. The handle arms 222 are connected to each other at the handle spring 224, and cross each other at an end opposite the handle spring 224. The handle spring 224 may be rounded and include a plurality of teeth such that when pressure is applied to the handle arms 222 to bring the handle arms 222 closer to each other, a space between the jaws 210 widens.

The jaws 210 includes a pair of necks 212 extending from the handle arms 222 at the crossing portion 225, and a pair of tips 214. The necks 212 each include a straight portion 212A and a bent portion 212B. The straight portions 212A of each of the necks 212 extend from the crossing portion 225 of the handle arms 222 away from each other the necks 212 bend at the bent portion 212B such that the tips 214 face each other, as illustrated in FIG. 16. The jaws 210 may have a suitable length (or there may be a suitable distance from the tips 214 to the crossing portion 225) such that the introducer 200 is spaced from the adhesive applicator 10 when the introducer 200 is used to remove the lens 1 from the adhesive applicator 10.

The handle arms 222 extend from the handle spring 224 and in a relaxed state, the handle arms 222 extend slightly away from each other as they extend from the handle spring 224. The handle arms 222 include a straight portion 222A and a bent portion 222B. The straight portion 222A is connected to (or extends from) the handle spring 224. The handle arms 222 are bent at the bent portion 222B and extend toward each other and cross each other at a crossing portion 225. The handle arms 222 may include a first handle arm 223A and a second handle arm 223B. The first handle arm 223A may include a slot 226 at the crossing portion 225, and the second handle arm 223B may be inserted through the slot 226 at the crossing portion 225. The second handle arm 223B may have a reduced width at the slot 226 and the slot 226 and the portion of the second handle arm 223B that is inserted through the slot 226 may be in a clearance fit configuration. When pressure is applied to the handle arms 222, the second handle arm 223B may move through the slot 226.

A gap 216 is defined between the tips 214. The gap 216 between the tips 214 increases when pressure is applied to the handle arms 222. In some embodiments, when no pressure is applied to the handle arms, the gap 216 between the tips may correspond to a width of the lens 1. As such, when pressure is applied to the handle arms 222, the gap 216 widens, allowing the jaws 210 to spread wider than the lens 1. When pressure is released, the gap 216 shortens again, and can provide a light clamp around the lens 1.

According to one or more embodiments, the tips 214 each define an indentation 215 at a lower portion thereof. The indentations 215 are formed to allow for improved contact with the lens 1 when the introducer 200 clamps the lens 1. The indentations 215 are offset from an edge of the introducer 200, to allow for the tips 214 to slide around and clasp the lens 1.

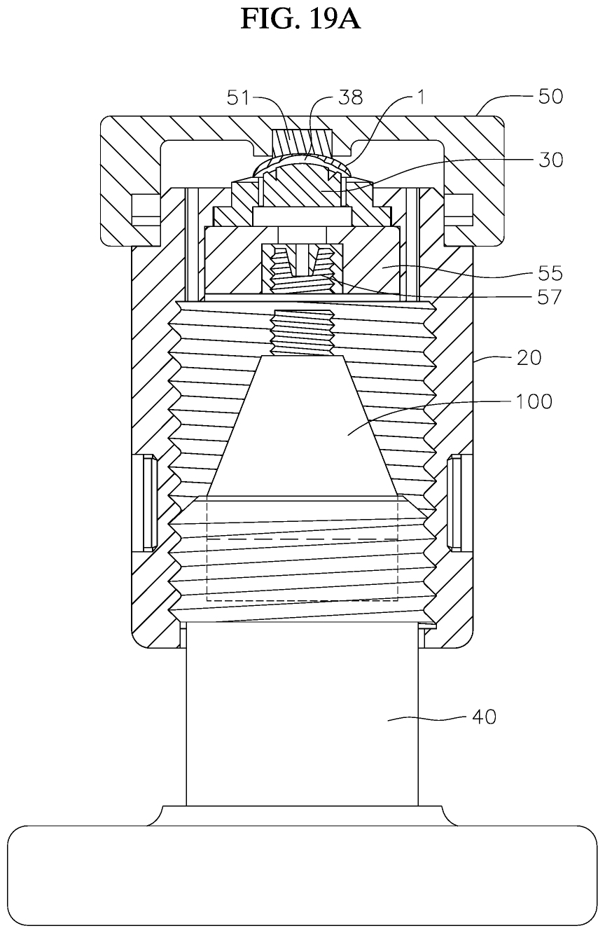

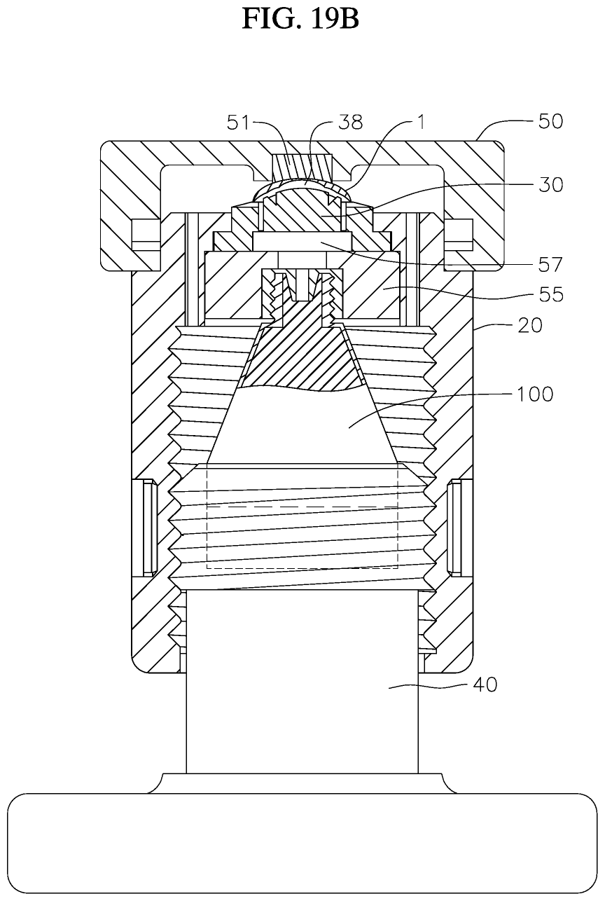

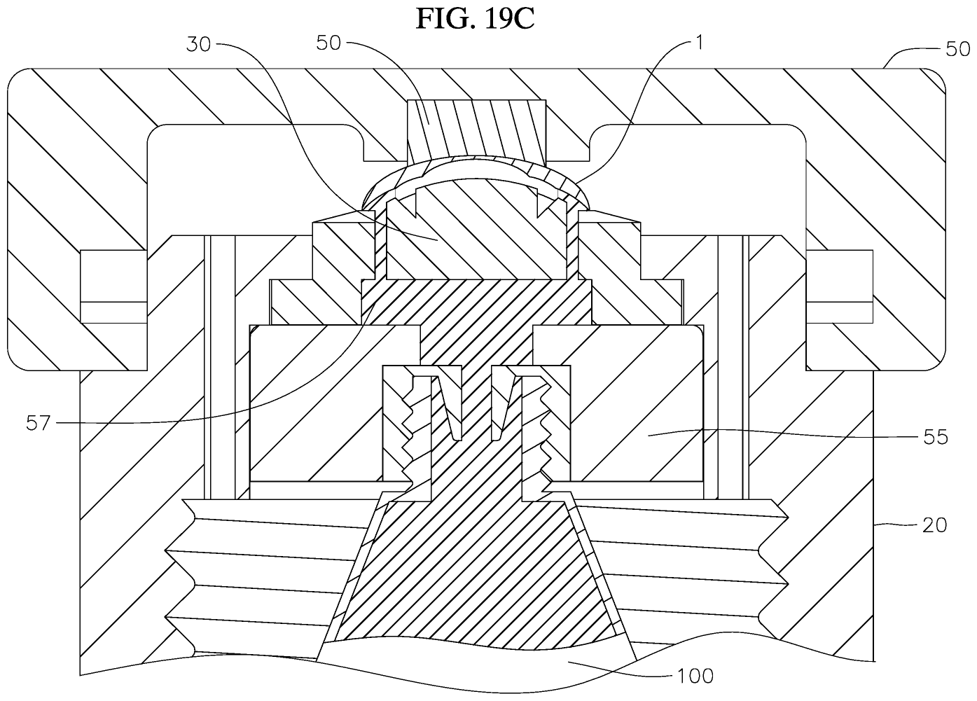

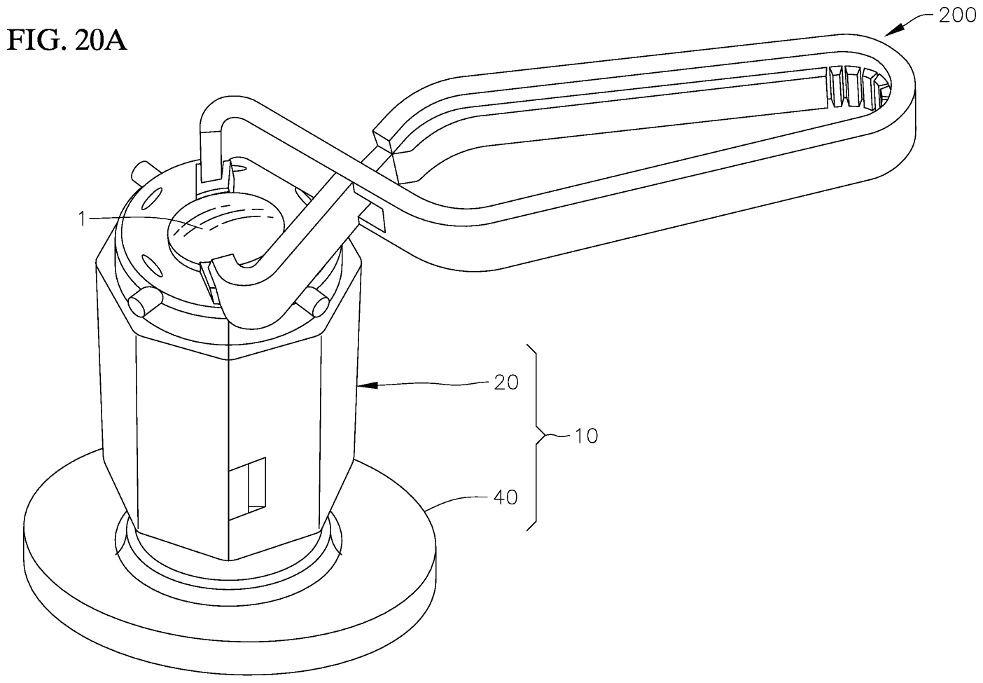

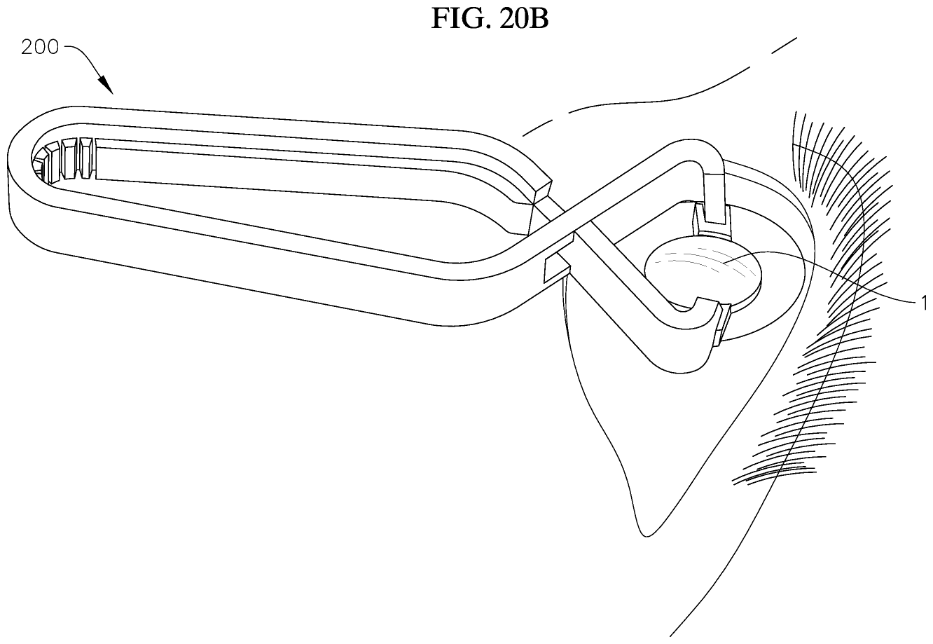

Referring to FIGS. 19A-19C, one or more embodiments of the present disclosure are directed to a method of extruding a fluid in a ring shape. According to one or more example embodiments, adhesive may be extruded onto the lens 1 using the adhesive applicator 10. Referring to FIGS. 20A-20B, one or more embodiments of the present disclosure are directed to a method of adhering the lens 1 to an eye. Referring to FIG. 19A, the adhesive container 100 is oriented such that the crimped portion 130 of the adhesive container 100 is inserted into the groove 47 of the plunger 40. The lens 1 is placed on the nozzle 30 (e.g., is placed above the nozzle 30 and rests on the locating tabs 39D). The gap 38 is formed between the lens 1 and the nozzle 30. The cap 50 is secured onto the body 20 (e.g., is secured onto the bayonet portion 23 by mating the slots 52 with the bayonet pins 26 and twisting), The pad 51 contacts the outer wall 2 of the lens 1. The pad 51, the locating tabs 39D, and the nozzle lands 39C prevent the lens 1 from shifting its position on the nozzle 30, or reduce the likelihood thereof.

Referring to FIG. 19B, the plunger 40 and the adhesive container 100 are driven toward the nozzle 30. For example, the body 20 and the plunger 40 may be rotated relative to each other (e.g., the body 20 may be rotated about the plunger 40 and/or the plunger 40 may be rotated relative to the body 20) to drive the plunger 40 and the adhesive container 100 toward the nozzle 30. As the adhesive container 100 moves toward the nozzle 30, and the nut 55, an outer surface of the adhesive container nozzle 122 may contact the inner wall of the stopple 57. In some embodiments, the adhesive container nozzle 122 is threaded and the inner wall of the stopple 57 is threaded such that the adhesive container nozzle 122 and the stopple 57 mate with each other. As the plunger 40 and the adhesive container 100 are rotated, the nut 55 prevents the stopple 57 from rotating (or reduces the likelihood thereof). As the adhesive container nozzle 122 moves upwardly toward the nozzle 30, the film 124 on the adhesive container nozzle 122 contacts the seal breaker 59, puncturing the film 124 and exposing the adhesive inside the adhesive container 100 to the outside.

Referring to FIG. 19C, when the adhesive container nozzle 122 is at the top of the stopple 57, as the plunger 40 continues to drive the adhesive container 100 toward the nozzle 30, the adhesive container 100 begins to compress, extruding adhesive through the stopple opening 58. The adhesive then flows through the stopple opening 58 into the inlet port 33. The adhesive then flows from the inlet port through the channel 36 upwardly toward the lens 1. The adhesive is applied to the lens 1 in a circular configuration. Excess adhesive may flow away from the lens 1 through the overflow channels 39A. The adhesive is applied to the lens 1 at the peripheral portion 4C in a circular shape.

Referring to FIG. 20A, while a user holds the handle 220 of the introducer 200, the jaws 210 of the introducer 200 are slid through the introducer ways 39B of the nozzle 30 toward the lens 1. The user may apply pressure to the handle 220 to widen the gap 216 between the jaws 210 around the lens 1. The user may then release pressure on the handle 220 to decrease the gap 216 as the indentations 215 on the jaws 210 contact the lens 1 (e.g., an edge of the lens 1). As such, the introducer 200 may then be utilized to lift the lens 1 from the adhesive applicator 10.

Referring to FIG. 20B, the introducer 200 may then be used to adhere the lens 1 to an eye (e.g., to a cornea of an eye). The user may align the lens 1 with the cornea or other region of the eye, position the lens 1 on the eye, and then apply pressure to the handle 220 of the introducer 200 to release the lens 1 from the introducer 200. As such, the adhesive applicator 10 applies adhesive to the lens 1, which is then adhered to an eye.

The preceding description has been presented with reference to various embodiments of the present disclosure. Persons skilled in the art to which this disclosure pertains will appreciate that alterations and changes in the described structures and methods of construction can be practiced without meaningfully departing from the principles, spirit, and scope of this disclosure.

While this disclosure has been described in detail with particular references to some exemplary embodiments thereof, the exemplary embodiments described herein are not intended to be exhaustive or to limit the scope of the disclosure to the exact forms disclosed. Persons skilled in the art and technology to which this disclosure pertains will appreciate that alterations and changes in the described structures and methods of assembly and construction can be practiced without meaningfully departing from the principles, spirit, and scope of this disclosure, as set forth in the following claims.

What is claimed is:

1. An applicator comprising:
a body defining a cavity configured to receive a container;
a nozzle at an upper portion of the body, the nozzle comprising:
an outer nozzle portion having a circular shape and defining a plurality of overflow channels;
an inner nozzle portion having a circular shape and spaced from the outer nozzle portion by a circular channel, the inner nozzle portion comprising a ridge around a periphery of the inner nozzle portion and a cleft defining the ridge, the cleft and the ridge each having circular shapes; and
a plurality of connectors extending through the channel and connecting the inner nozzle portion to the outer nozzle portion,
wherein the overflow channels extend radially outward from the channel; and
a plunger comprising a shaft and a base, the shaft being movable within the cavity and configured to guide the container toward the nozzle.

2. The applicator of claim 1,
wherein the body comprises a barrel portion, and
wherein an inner wall of the barrel portion has female threads and wherein at least a portion of an outer wall of the plunger has male threads, the male threads of the plunger mating with the female threads of the barrel portion.

3. The applicator of claim 2, further comprising a nut below the nozzle, the nut being in a clearance fit configuration with the body, and configured to resist rotation by the plunger when the plunger is rotated.

4. The applicator of claim 3,
wherein the nut comprises a stopple comprising a seal breaker at a lower portion of the stopple, the seal breaker having a first thickness at an uppermost portion of the seal breaker and a second thickness at a lowermost portion of the seal breaker, the first thickness being greater than the second thickness, and
wherein the seal breaker defines a stopple opening at a radial center of the stopple, the stopple opening being in fluid communication with the channel of the nozzle.

5. The applicator of claim 4, wherein an inner wall of the stopple comprises female threads, the female threads being configured to mate with male threads of a container nozzle of the container.

6. The applicator of claim 1, wherein the nozzle further comprises an inlet port in fluid communication with the channel and between the channel and the plunger along a longitudinal direction of the applicator, wherein the inlet port has a diameter that is greater than or equal to an inner diameter of the outer nozzle portion.

7. The applicator of claim 1, wherein the body comprises a plurality of vents defined in a top surface of the body, the vents being in fluid communication with the cavity.

8. The applicator of claim 1,
wherein the body comprises a barrel portion and a bayonet portion, the bayonet portion comprising a plurality of bayonet pins protruding from the bayonet portion,
and wherein the applicator further comprises a cap, the cap defining a plurality of slots configured to mate with the bayonet pins.

9. The applicator of claim 8, wherein the cap comprises a pad at a radial center thereof, the pad extending toward the nozzle when the cap is mated with the bayonet portion.

10. A contact lens comprising:
an outer wall have a substantially continuous curvature; and
an inner wall, the inner wall comprising a first portion having a first curve radius, a secondary portion having a second curve radius, and a peripheral portion having a third curve radius, the second curve radius being different from the first curve radius and the third curve radius.

11. The contact lens of claim 10, wherein the second curve radius is greater than the first curve radius.

12. The contact lens of claim 10, wherein the third curve radius is greater than the second curve radius.

13. The contact lens of claim 10, wherein the contact lens has a center thickness at an apex of the contact lens of between about 0.10 mm and about 0.14 mm.

14. A corneal graft and adhesive applicator kit, the kit comprising:
a contact lens comprising:
an outer wall have a substantially continuous curvature; and
an inner wall, the inner wall comprising a first portion having a first curve radius, a secondary portion having a second curve radius, and a peripheral portion having a third curve radius, the second curve radius being different from the first curve radius and the third curve radius;
an adhesive container comprising a body and an orifice, the adhesive container containing an adhesive;
an adhesive applicator comprising:
a body defining a cavity configured to receive an adhesive container;
a nozzle at an upper portion of the body, the nozzle comprising:
an outer nozzle portion having a circular shape and defining a plurality of overflow channels;
an inner nozzle portion having a circular shape and spaced from the outer nozzle portion by a circular channel, the inner nozzle portion comprising a ridge around a periphery of the inner nozzle portion and a cleft defining the ridge, the cleft and the ridge each having circular shapes; and
a plurality of connectors extending through the channel and connecting the inner nozzle portion to the outer nozzle portion,
wherein the overflow channels extend radially outward from the channel; and
a plunger comprising a shaft and a base, the shaft being movable within the cavity and configured to guide the adhesive container toward the nozzle; and
an introducer comprising jaws and a handle.

15. The kit of claim 14, wherein the handle of the introducer comprises a first handle arm, a second handle arm, and a handle spring connecting the first handle arm and the second handle arm.
